(19) United States
(12) Patent Application Publication
(10) Pub. No.: US 2003/0175994 A1
(43) Pub. Date: Sep. 18, 2003

(54) Geometrically efficient particle agglutination, particularly to detect low affinity

(76) Inventor: Yoram Palti, Haifa (IL)

Correspondence Address: Paul F Fehlner, Darby & Darby, Post Office Box 5257, New York, NY, 10150-5257, US

(21) Appl. No.: 10/380,378

(22) PCT Filed: Oct. 1, 2001
(86) PCT No.: PCT/IB01/01829

(51) Int. Cl. G01N 33/546

(52) U.S. Cl. 436/533

(57) ABSTRACT

Compositions and methods for detection of binding interactions, particularly low affinity binding interactions, are provided. The methods and compositions are suitable for diagnostic assays, and other assays for the presence of low affinity binding compounds in a sample. In one exemplary embodiment, the method includes detecting agglutination of geometrically regular particles to which a molecule which is a member of a binding pair is covalently conjugated.

### CROSS-REFERENCE TO RELATED APPLICATION

This application claims the benefit of U.S. provisional application Serial No. 60/238,061, filed Oct. 5, 2000, and which is hereby incorporated by reference in its entirety.

### FIELD OF THE INVENTION

The present invention relates to compositions and methods for detection of binding interactions, particularly low affinity binding interactions. The methods and compositions are suitable for diagnostic assays, and other assays for the presence of low affinity binding compounds in a sample.

### BACKGROUND OF THE INVENTION

Immunoassays involving the agglutination of finely divided particles have been known for some time. In such assays, a liquid sample containing the analyte under assay is mixed with finely divided particles bearing a reagent (normally present as a coating thereon), and the particles agglutinate to an extent dependent on the presence and/or amount of analyte in the sample. Thereby the presence and/or amount of the analyte can be determined. In particular, agglutination of latex particles has been applied extensively to the detection of proteins and haptens (Bangs, Uniform Latex Particles, Seradyn Inc.: Indianapolis Ind., 1984, pp. 51-58; see PCT Publication WO 92/04469), as well as for detecting nucleic acid sequences (see PCT Publication Nos. WO 87/05334 and WO 92/04469).

There are various ways in which such assays can be effected in practice. In one well known procedure for the assay of an antigen (such as alpha-fetoprotein), the liquid sample is mixed with a known amount of latex particles, which carry an antibody to the antigen. The antibody and antigen bind to form complexes, thus agglutinating the particles to an extent in proportion (but not usually direct proportion) to the amount of antigen present. The extent of agglutination may then be measured, preferably by selectively counting the unagglutinated particles.

One particular use of agglutination immunoassays is in the assay of human body fluids, such as serum for example. It has been found, however, that such fluids commonly contain, in addition to the particular analyte under assay, other materials which either cause or inhibit agglutination and so interfere in the assay, causing errors in the quantitative results. Interference of this type cannot properly be offset by comparing the assay result with the result of a similar assay made on blank serum (viz. serum not containing the analyte in question), since the blank serum may well not be truly representative of the particular patient's serum containing the analyte under assay.

In particle agglutination assays, the commonest and most widely used particles are latex particles, which consist normally of a synthetic polymeric material such as polystyrene. Other types of particles, such as various clays, can be used but are not so widely employed. A reagent is attached to the particles, the reagent being a molecule which takes part in the assay reaction. In cases where the reagent is itself a material which can form a coating, e.g., an immunoglobulin, then a reagent coating may be applied to protect the underlying particle core. Alternatively, and in other cases where the reagent cannot form a protective coating, e.g., where the reagent is an antigen or hapten, an inert coating is applied to the particles to cover the core. For various reasons, the most common such inert coating material is a protein, such as bovine serum albumin. The reagent may be attached to the core or to the coating. Thus, in particle agglutination assays, the particles commonly have a protein coating which is either inert to the immunospecific reaction of the assay, or constitutes a reagent in that reaction.

Chaotropic agents are known per se and have a number of properties, among which is breaking or weakening non-covalent bonds such as hydrogen, electrostatic and hydrophobic bonds in principle, therefore, they can reduce weak protein-protein interactions, i.e., dissociate electrostatic and hydrogen bonds. Thus, it has been shown that they also have the effect of weakening the bonding in antibody: antigen complexes. Since most agglutination immunoassays are based on antibody: antigen complex formation, it would be advantageous to enhance the specific binding interaction while at the same time disrupting non-specific interactions with a chaotropic reagent (see, e.g., U.S. Pat. No. 4,362,531).

Assays involve two components: specificity and sensitivity. Specificity relates to accuracy: does the assay detect the presence of the analyte, particularly to the exclusion of other analytes. Assays with better specificity have lower percentage of false positives. Sensitivity relates to the concentration threshold at which an analyte can be detected: greater sensitivity permits detection of smaller amounts of analyte. Assays with better sensitivity have a lower percentage of false positives. There is a need in the art to achieve high values of both specificity and sensitivity in assay systems.

### SUMMARY OF THE INVENTION

The present application relates to a method for detecting binding of two or more molecules, particularly low affinity binding interactions. The method comprises detecting agglutination of geometrically regular particles. One molecule, which is a member of a binding pair, is covalently conjugated to the particle. Agglutination is detected in the presence of a second molecule, which is a second member of the binding pair. Preferably the geometrically regular particle is cubic or flat. In specific embodiments, one binding molecule is an antibody and the second binding molecule is an antigen.

The application further relates to a composition comprising geometrically regular particles to which a molecule which is a member of a binding pair is covalently conjugated. The application further relates to a method for preparing such a composition. This method comprises conjugating a molecule which is a member of a binding pair to particles of material that have a geometrically regular shape. Such particles of material may be obtained by extrusion through a shaped die and cutting at regular intervals. In a specific embodiment, the shaped die is square shaped. Alternatively, the particles can be molded. In yet another embodiment, the particles are etched of micro-machined or cut from a block material or a sheet.

Various materials can be used in the formation of the geometrically regular particles, including polyacrylamide, agarose, latex, starch or a starch derivative, silica, silicon, polyamide or any other polymeric plastic material, or a ferromagnetic or paramagnetic material, to mention a few such examples.

This invention may be better understood by reference to the following drawings, detailed description, and examples.

### BRIEF DESCRIPTION OF THE DRAWINGS

FIGS. 1A and 1B are schematic drawings illustrating interaction of agglutinated particles with (A) a cubic shape and (B) a spherical shape;

FIGS. 2A and 2B are cross sectional views of stack of agglutinated plates with (A) showing the open position and (B) showing the closed position;

FIGS. 3A and 3B illustrate stacks of connected agglutinated plates with (A) illustrating the open position and (B) illustrating the closed position;

FIGS. 4A and 4B are sectional views of stacks of connected agglutinated plates in the open position with (A) illustrating the front view and (B) illustrating the side view;

FIGS. 5A and 5B are cross sectional views of a bellow-shaped agglutination strip with (A) showing the open position and (B) showing the closed position;

FIGS. 6A and 6B illustrate the bellow-shaped agglutination strip with (A) showing the front view and (B) showing the side view;

FIGS. 7A and 7B illustrate fan-shaped geometrically regular plates with (A) showing the open position and (B) showing the side view;

FIGS. 8A ,8B, and 8C illustrate geometrically regular shaped agglutination particles (e.g. cubic particles) with (A) illustrating the geometrically regular agglutination particles in suspension, (B) illustrating the geometrically regular shaped agglutination particles before the liquid sample containing an analyte is added, and (C) illustrating the geometrically regular shaped agglutination particles after the liquid sample containing an analyte is added;

FIGS. 9A, 9B, and 9C illustrate geometrically regular shaped agglutination particles using ferromagnetic material with (A) illustrating the geometrically regular shaped agglutination particles using ferromagnetic material in suspension before the liquid sample containing an analyte is added, (B) illustrating the enhancement of agglutination using a magnet to temporarily clump the agglutinated particles in suspension, and (C) the agglutinated ferromagnetic particles after the magnet is removed;

FIGS. 10A, 10B, and 10C illustrate magnetic agglutinated particles with (A) illustrating magnetic agglutinated particles (in demagnetized state) suspended in solution while the liquid sample containing an analyte is added, (B) illustrating the magnetic agglutinated particles aligning in order while magnetized, and (C) illustrating the magnetic agglutinated particles demagnetized;

FIGS. 11A and 11B illustrate a strategy for detecting agglutination with (A) illustrating an electrical means which measures changes in impedance due to agglutination of electrically resistant agglutinated particles and (B) illustrating an optical means which measures the intensity of light reflected from particles as a measure of the occurrence of agglutination (e.g. fluorescent geometrically regular shaped particles).

### DETAILED DESCRIPTION

As noted above, the present compositions and methods provide for detection of an analyte in a test sample. In particular, the invention, by virtue of the unique geometric arrangement of binding members, permits achievement of high levels of sensitivity and specificity for a binding interaction. Binding interaction can be readily detected by virtue of the change in physical properties of the suspension of geometrically regular particles upon stabilized agglutination.

The present compositions and methods are particularly useful for detecting binding in low affinity situations, where the aggregate binding energy of a large number of interactions overcomes the otherwise weak binding of individual binding members in a low affinity interaction. Thus, high specificity, low affinity binding is elevated to a high degree of sensitivity in accordance with the present disclosure.

Specific low affinity binding conditions include those in which denaturant is added to the test sample in order to interfere with non-specific binding interactions. The presence of the denaturant will weaken strong, specific binding activity. However, as noted above, the overall decrease in each individual binding interaction is overcome by the aggregate binding energy, which in turn is enhanced by the regular orientation of the geometrically regular particles. Specificity is achieved in this circumstance in part by virtue of a reversal of the denaturing effect upon binding of the binding members, i.e., the analyte and the binding molecule specific for the analyte. Thus non-specific binding interactions are unlikely to achieve the same conformation because they will not have the same energy. Thus the presence of denaturant will tend to have a greater disruptive effect in non-specific binding interactions.

The term “test sample”, as used herein, refers to a material suspected of containing an analyte. A sample can be used directly as obtained from the source or following a pretreatment to modify the character of the sample. The test sample can be derived from any biological source, such as a physiological fluid, including blood, saliva, ocular lung fluid, cerebral spinal fluid, sweat, urine, milk, seminal fluid, mucous, sinovial fluid, peritoneal fluid, amniotic fluid, or the like. Alternatively, the test sample can be from cell culture fluid or fermentation culture fluid. In yet another embodiment, a test sample is from an environmental source, such as treated or untreated water, sewage, ground water, or the like. Other samples include food products, and beverages. The test sample can be pretreated, by removing cells from blood or culture fluid, or to interactivate into variant proponents or by the addition of reagents such as buffers and extraction materials. Solid materials may be liquefied prior to testing in order to yield analyte.

The term “binding member”, as used herein, refers to a member of a binding pair, i.e., two different molecules where one of the molecules specifically binds to the second molecule through chemical or physical means. In addition to the well-known antigen and antibody binding pair members, other binding pairs include, as examples without limitation, carbohydrates and lectins, biotin and avidin or strepavidin, complimentary nucleotide sequences, complimentary peptide sequences, effector and receptor molecules, enzyme co-factors and enzymes, enzyme inhibitors and enzymes, enzymes substrates and enzymes, and any other similar molecules having a binding affinity that permits their association in a binding assay. Preferably, binding members bind with high specificity, even if that results in relatively low affinity, since the strategies of the present invention overcome individual weak binding through enhanced aggregate binding. An example of such a low affinity binding interaction is a cross-reactive binding interaction of an antibody for an analogous analyte. The details of preparation of antibodies and other binding pair members are well-known to those of skill in the art.

The term “analyte” or “analyte of interest”, as used herein, refers to the compound or composition to be detected or measured and which has at least one epitope per bind site for a binding member. It generally could be any substance for which there exists a naturally occurring binding member for which a binding member can be prepared. Analytes include, but are not limited to, toxins, organic compounds, proteins, peptides, carbohydrates, microorganisms, amino acids, nucleic acids, hormones, steroids, vitamins, drugs (including those administered for therapeutic purposes as well as those administered for illicit purposes), and metabolites or for antibodies to any of the above substances. The term analyte also includes any antigenic substances, haptens, allergens, macromolecules, or combinations thereof. Preferably the analyte is multivalent, i.e., contains more than one epitope for a binding member.

The term “analyte-analog”, as used herein, refers to a substance that cross-reacts with an analyte-specific binding member, although it may do so to a greater or lesser extent than it does to the analyte itself. The analyte-analog can include a modified analyte as well as a fragmented or synthetic portion of the analyte molecule, so long as the analyte-analog has at least one epitopic site in common with the analyte of interest. An analyte-analog can also be a closely related molecule to the analyte.

The term “solid support”, as used herein, embraces a particle with the appropriate sites for irreversible association of a binding member. A solid support is useful in preparation of geometrically defined particles for use in agglutination assays of the invention. Supports may consist of many materials, limited primarily by capacity for derivitization or irreversible association of the binding member. Examples of support material include, but are not limited to, glass, latex, cross-linked polystyrene or similar polymers, colloidal metal particles, agarose, polyacrylamide, starch or starch derivatives, silicon, polyamide, and the like. Additional solid phase materials include, without limitation, polymers of styrene, substituted styrenes, naphthalene derivatives, acrylic and methacrylic acids, acrylamide and methacrylamide, polycarbonate, polyesters, polyamides, polypyrrole, polypropelene, polytetrafluoroethylene, polyacrylomitrile, polycarbonate, polyesters, polyamides, polypyrrole, polypropelene, polytetrafluoroethylene, polyacrylonitrile, polycarbonate, amino aromatic acids, aldehydes, proteanatious materials (such as gelatin and albumin), polycyclides (including dextran), and copolymers of such polymeric materials. Other examples, natural, synthetic, or naturally occurring materials that are synthetically modified can be used as a solid base material including polysaccharides, e.g., cellulose materials such as paper and cellulose derivatives such as cellulose acetate and nitrocellulose; organic materials such as deactivated alumina, or other inorganic finely divided material uniformly dispersed in a porous polymer matrix, with polymers such as vinyl chloride, vinyl chloride polymer with propylene, co-polymer with vinyl acetate; porous gels such as silica gel, natural polymers such as dextran, and gelatin; polymeric films such as polyacrylades; protein binding membranes; and the like. Chemically reactive groups for use with such solid supports may be derivitized, commonly used for solid state synthesis of oligomers, or used for covalent or non-covalent association of binding members.

In addition, magnetic, diamagnetic, or paramagnetic materials can be used as solid supports. Magnetically attractable materials include ferromagnetic, ferrimagnetic, paramagnetic, and supermagnetic materials. The term “ferromagnetic” is generally used to describe materials which are attracted to a magnet to a high degree, and which typically become permanently magnetized upon exposure to a magnetic field. Ferromagnetic materials may also be reduced in particle size so that each of the particles is a single domain. Such paramagnetic material may be referred to as “superparamagnetic,” characterized by the absence of any permanent measurable magnetization. Suitable magnetically repulsed materials include diamagnetic materials including, but not limited to, organic polymers such as polystyrene. Suitable magnetically attractable materials include metals (e.g., iron, nickel, cobalt, chromium, and manganese), lanthanide series elements (e.g., neodymium, erbium), alloys (e.g., magnetic alloys of aluminum, nickel, cobalt, copper), oxides (e.g., Fe₃O₄, Fe₂O₃, CrO₂, CoO,NiO₂, Mn₂O₃), composite materials (e.g., ferrites), solid solutions (e.g., magnetite with ferric oxide), and composites (e.g., plastics impregnated with any of the foregoing). Preferred magnetic materials involve magnetite, ferric oxide (Fe₃O₄) and ferrous oxide (Fe₂O₃). Additional suitable paramagnetic and magnetic materials are described in PCT Publication No. WO 93/19371.

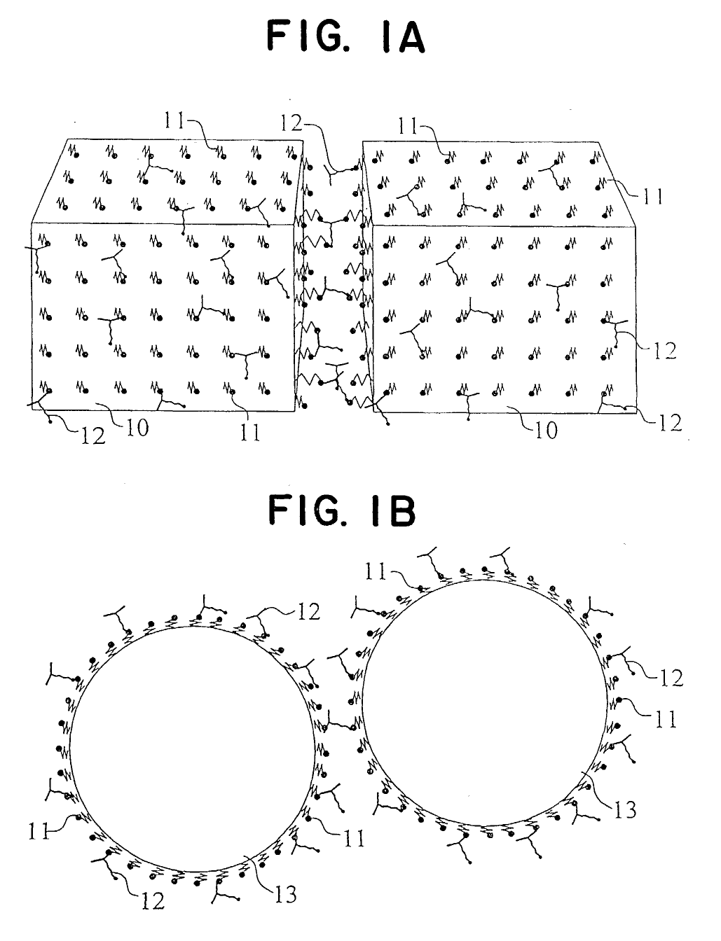

As used herein, the term “geometrically regular particles” refers to particles that are manufactured to have regular geometric shapes, comparable in size. The suitable shapes include, but are not limited to, regular polyhedrons, such as cubes and tetrahedrons, and in sheets of any desired shape, including circles, ovals, and other rounded shapes; regular and irregular tetrahedrons; squares or rectangles; irregular flakes; and any other such shape. Such flat particles can be packed to fill a whole volume and have extremely high surface-to-volume ratios. Preferred geometrically regular particles can be packed to fill the entire volume and have a high fraction of surface-to-surface contact area. Any geometries that provide for these features are suitable for implementation in the practice of the invention. Naturally, spherical or spheroidal particles will lack these features. Accordingly, the term “geometrically regular” specifically excludes spherical or substantially spherical, including irregular spherical, particles. FIG. 1 is a schematic of the interaction of the agglutinated particles, FIG. 1A showing a cubic embodiment of geometrically regular particles (10) and FIG. 1B showing spherical particles (13). FIG. 1 also illustrates the binding of antigen (or other analyte) (12) to antibody (or other binding pair member) (11) on the geometrically regular particles (10).

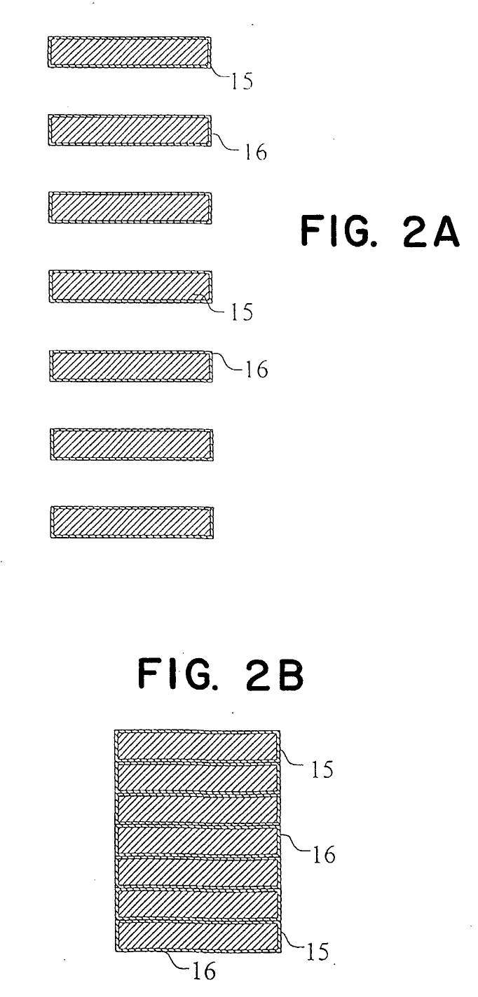

Flat particles contain a plate-shaped support (15) and an agglutinated coating (16) as shown in FIGS. 2A and 2B. FIG. 2A shows a cross sectional view of stacked agglutinated plates in the open position. FIG. 2B further shows a cross sectional view of stacked agglutinated plates in the closed position.

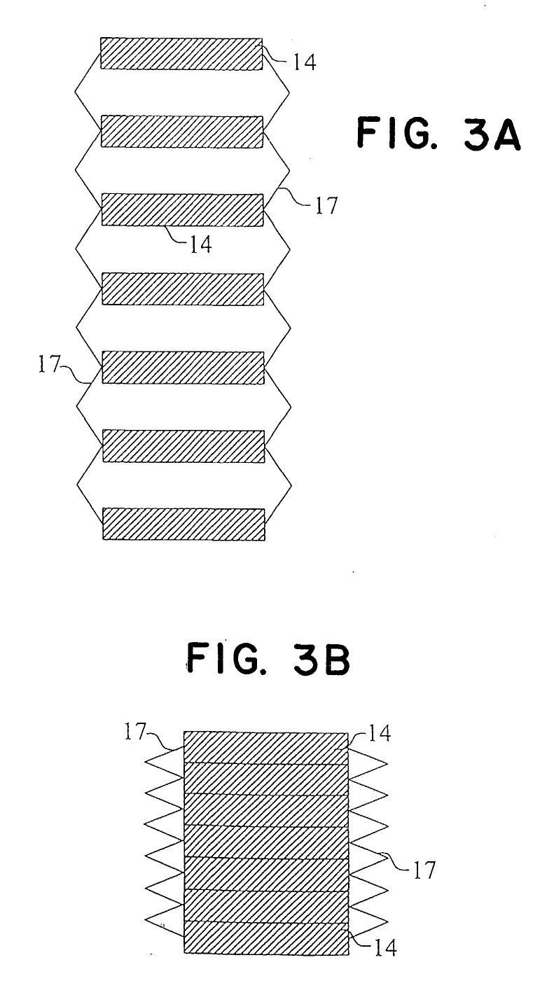

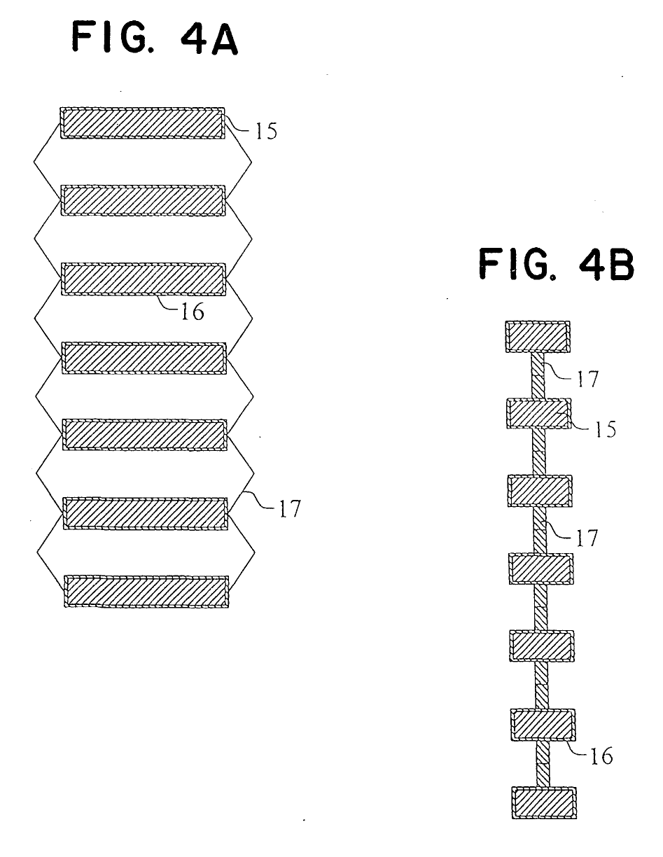

As shown in FIG. 3, the agglutinated plate (14) may be connected to another agglutinated plate (14) by means of a spring mechanism (17). FIG. 3A shows stacks of connected agglutinated plates in the open position and FIG. 3B shows stacks of connected agglutinated plates in the closed position. FIGS. 4A and 4B further illustrate the stacking feature with FIG. 4A showing a front cross sectional view of stacks of connected agglutinated plates in the open position and FIG. 4B showing a side cross sectional view of stacks of connected agglutinated plates in the open position.

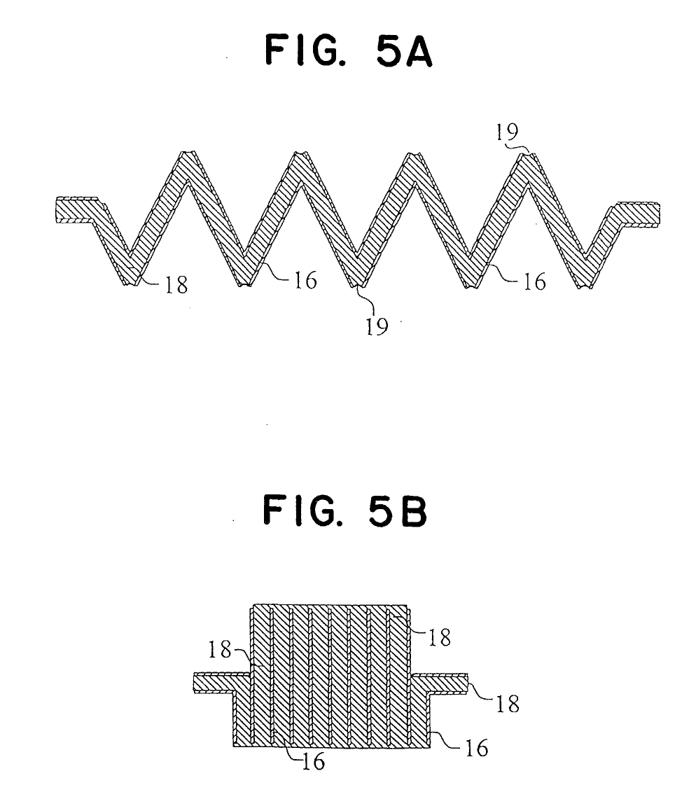

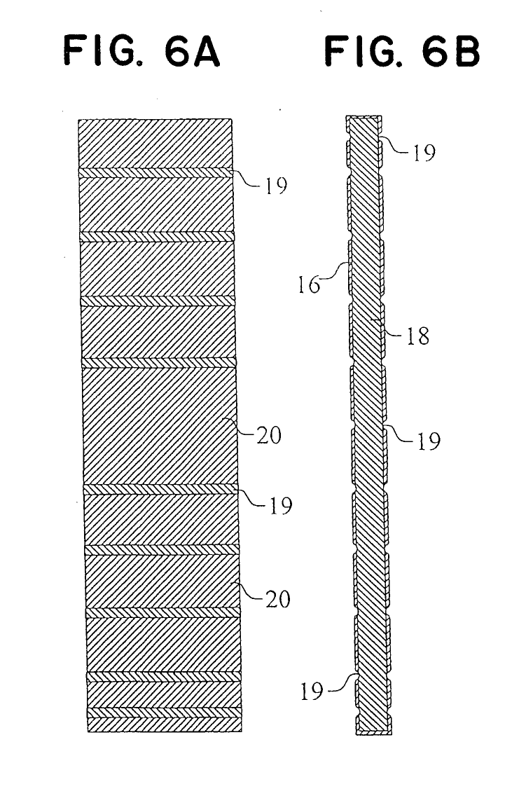

Flat particles with an agglutinated coating (16) may also be arranged in a bellow-shaped format with a grove to facilitate bending (19) and a bellow-shaped support (18) as shown in FIGS. 5A and 5B. FIG. 5A is a cross sectional view of the bellow-shaped agglutination strip in the open position. FIG. 5B is a cross sectional view of the bellow-shaped agglutination strip in the closed position. FIG. 6A is a front view of the bellow-shaped agglutination strip, illustrating the position of the agglutinated strip (20). FIG. 6B is a side view of the bellow-shaped agglutination strip.

An agglutinated plate (21) may attached to another agglutinated plate (21) in a fan-shaped format as shown in FIGS. 7A and 7B. FIG. 7A shows the fan-shaped geometrically regular plates in the open position, and FIG. 7B shows the fan-shaped geometrically regular plates in the closed position.

Preferably, the geometrically regular particles have roughly the same size. Another way to put this is that they have about the same volume. Volumes among geometrically regular particles naturally will vary within defined allowable variants, which would depend on manufacturing methods and the degree to which they can be controlled.

As used herein, the term “about” or “approximately generally means within 50% of a given value, preferably within 20%, more preferably within 10%, and more preferably still within 5%. Alternatively, particularly in biological assay systems, “about” can mean within an order of magnitude, preferably within 5-fold, and more preferably within 2-fold of a given value. Thus, these terms most preferably refer to a given value within an acceptable experimental range, e.g., to those of ordinary skill in the art.

Preparing Geometrically Regular Particles

The present invention provides numerous ways for preparing geometrically regular particles. Particles can be blown-injected in a mold or space, cut from blocks of material or sheets of material, compressed in the desired shape, extrusion through a shape die with cutting at regular intervals, or other methods known in the art. For example, silicon microparticles can be prepared by micromachining or etching particles such as for manufacturing microelectronic integrated circuits (see PCT Publication No. WO 99/41006). Flat supports, e.g., of plastic material, nitrocellulose, or even paper, can be cut using punches, dies, blades, or lasers, to yield agglutination particles.

In a specific embodiment, flat supports can be cut in a manner that permits folding to create a hollow support, e.g., akin to forming carton. Preferably the fold lines of the flat support are prepared by partially cutting the support where folds are required, i.e., by preparing grooves in the support.

An alternative method for preparing a hollow or lower density support is to coat a labile core with a support material, then remove the core. Cores can be prepared from differentially soluble material and extracted with solvent; or from labile material that can be dried; or from low melting material that can be melted. Various materials, such as hydrogels, which provide support but have very low density, can act as cores as well. Hydrogel materials include, but are not limited to, proteins (e.g., collagen, gelatin, albumin, etc.), polyethylene glycol, charged or neutral polysaccharides (e.g., hyaluronic acid, xanthates, alginates, guar gum, agarose, etc.) and starches.

Once the geometrically regular particle is prepared, it should be irreversibly conjugated with a binding member, or more than one binding member specific for different binding regions of an analyte (so that a monovalent analyte will nevertheless bind to multiple geometric particles). Association of the binding member with the particle is achieved by any techniques known in the art, including non-covalent and covalent associations.

Non-covalent links generally involve non-specific absorption binding process. A solution comprising the binding member is contacted with the solid state support under conditions that result in reversible absorption of the binding member to the solid support. Generally such conditions include a physiologically buffered solution, the absence of other molecules that would compete for non-specific binding sites on the solid state support, humidified conditions (humid chamber or sealing) to prevent evaporation of the coating solution, with incubation at an appropriate temperature, e.g., room temperature, for a sufficient period of time to permit irreversible binding to occur, e.g., at least about one hour and preferably more than one hour or overnight. Such binding interactions readily occur with solid state support such as plastic, glass, nitrocellulose, nylon, and similar materials.

Covalent binding usually requires activation of a functional group on the solid state support. Affinity chromatography media, which comprise a matrix that is activated for attachment of a ligand, possibly via a spacer, can be used if modified to adopt a geometrically regular shape (see Biochemicals and Reagents For Life Science Research, Sigma, St. Louis, Mo.). Many of these chemistries can be adapted to other solid state supports as well. Cyanogen bromide activation, epoxy activation, nitrophenylchloroformate, and N-hydroxysuccinimide chloroformate, to mention such reagents, can be used to covalently link the binding member to the matrix. Alternatively, other well known bi-functional cross-linking agents, such as activated carbonyls (ketones, aldehydes, and hydrides, acids), can be used. Suitable substrates for activation include, but are not limited to, agarose, acrylamide, acrylic beads, magnetic particles, cellulose, nitrocellulose, celite, and polystyrene.

Once prepared, the geometrically regular particles coated with the binding member can be stored. Preferably, storage conditions are such that the binding activity is preserved. Such storage conditions include in buffered solutions, possibly with preservatives or stabilizing proteins like serum albumin. Alternatively, the solid support can be lyophilized and stored in dry form. In yet another embodiment, the solid support, either dry or in solution, can be frozen in order to preserve it.

Assay Methods

The assay methods of the present invention may be applied to any suitable assay form involving binding pair members including, but not limited to, binding members (binding member and analyte) described above. Preferably, the assay methods of the invention will be employed to detect binding between binding pair members that interact with low affinity. In general, the agglutination assays of the invention will depend on a sort of sandwich assay, in which a binding member is irreversibly (covalently or non-covalently) associated with a geometrically regular particle. The binding member will bind to an analyte present in a sample.

In a direct agglutination format, analytes should be chosen or modified so that they can interact with more than one binding member or different particles. Alternatively, various modifications are available but to permit oligomerization of analytes so that they will bind to at least two binding members irreversibly associated with the particle.

In indirect binding formats (or agglutination inhibition formats) binding reactions between the binding members are disrupted by the presence of monovalent “artificial” analyte in the sample. When analyte is absent from the sample, binding of the multivalent artificial analyte to binding members promotes agglutination. When analyte is present, it competes with the “artificial” analyte for binding members, thus disrupting the adhesive forces and preventing agglutination. Thus, the indirect format yields the opposite observation of direct.

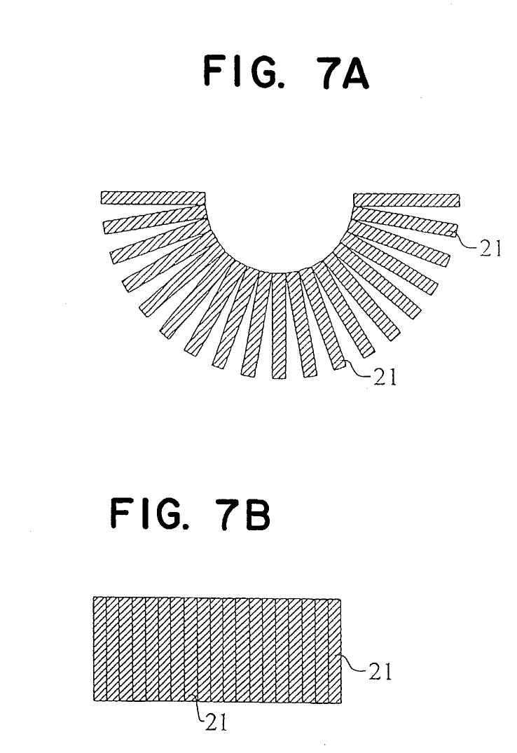

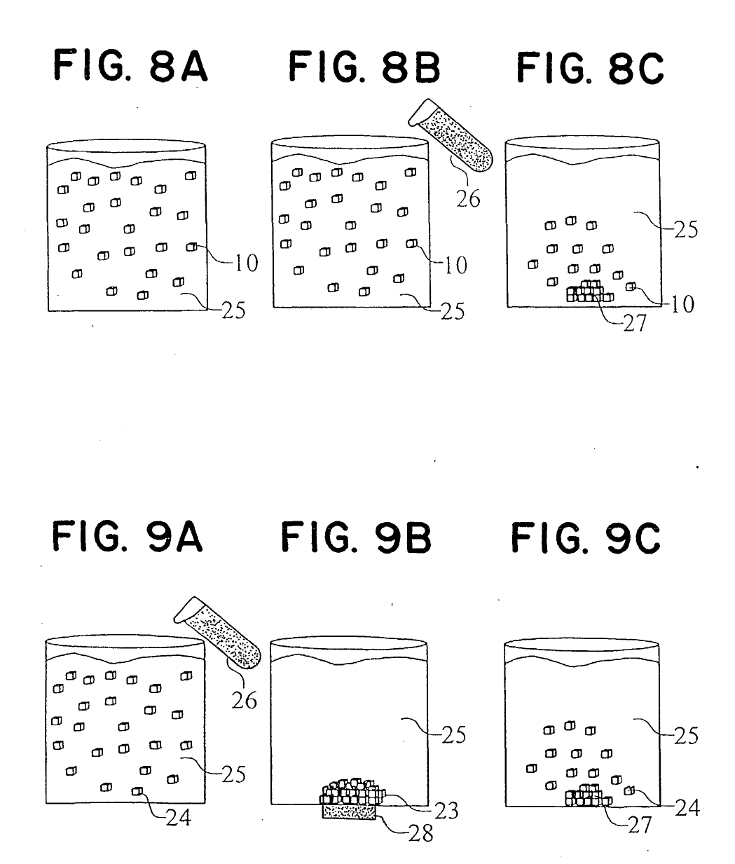

For example, FIG. 8A refers to the geometrically regular shaped particles (e.g. cubic particles) (10) in an appropriate buffer solution (25). The liquid sample containing an analyte (26) is added to a suspension of geometrically regular particles (10) in an appropriate buffer solution (25) (FIG. 8B). Preferably buffer solutions are physiologically buffered, but any solution that permits specific binding interaction of the binding members is permitted. As noted above, the assays system of the invention permits detecting specific binding interactions under denaturing conditions, which suppresses non-specific interactions. Denaturing conditions include but are not limited to the presence of chaotropic (e.g., urea, guanidine, hydrochloride, and the like); organic solvents (e.g., alcohols, polyalcohols, etc.); organic polymers (e.g., polyethylene glycol); heat; and salts. After the liquid sample containing an analyte (26) is added to the buffer solution (25) containing the geometrically regular particles (10), binding occurs resulting in agglutinated geometric regular particles (27) as seen in FIG. 8C.

In order to obtain optimal packing, the reaction mixture (i.e., the suspension of particles and analyte), may be shaken or vibrated. Discs may be somewhat more problematic, as they may form alternating geometries. However, the discs may be arranged in parallel rows with spaces between them or joined together as shown in FIGS. 3-7 to constrain their degrees of freedom. The discs can be compressed after a sufficient time has passed to react with the solution which possibly contains analytes is added to the spaces, and the adhesive properties of the discs can then be measured to determine whether there is a specific binding interaction that stabilizes the association of the discs. In the absence of such a binding interaction, the discs will separate readily. However, in the presence of the specific binding interaction, the compressed discs will remain in a compressed state. Another embodiment of the present invention involves the use of a ferromagnetic material. FIG. 9A represents geometrically regular shaped particles made from a ferromagnetic material (24) suspended in an appropriate buffer solution (25) before the liquid sample containing an analyte (26) is added. Agglutination is enhanced using a magnet (28) which attracts the clumped ferromagnetic agglutinated particles (23) in the buffer solution (25) as seen in FIG. 9B. Removal of the magnet, as seen in FIG. 9C causes separation of the clumped ferromagnetic agglutinated particles (23) from the agglutinated geometrically regular particles (27).

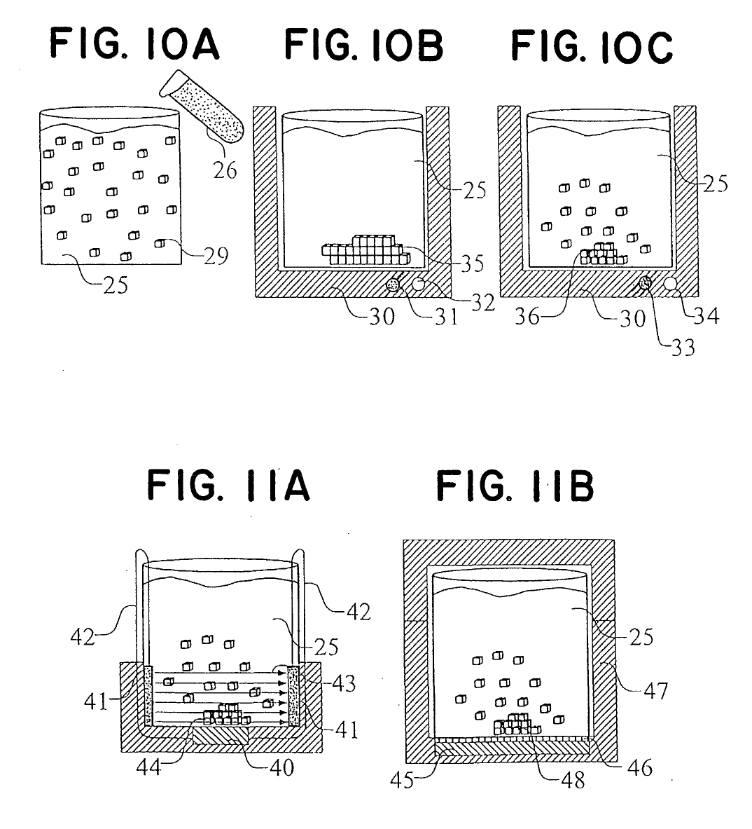

In a specific embodiment, the particles are magnetic, such that magnetic forces align them. FIG. 10A shows magnetic geometrically regular particles (29) suspended in an appropriate buffer solution (25) while the liquid sample containing an analyte (26) is added. Using a magnetizer/demagnetizer unit (30) with the magnetized option activated (31) and the demagnetized option deactivated (32), the magnetic agglutinated particles align (35) as seen in FIG. 10B. After magnetic agglutination, the particles can be demagnetized so they will fall apart unless they are bound together by chemical forces, i.e., the specific binding interaction of the binding members. FIG. 10C further illustrates by showing the magnetize option deactivated (33) and the demagnetize option activated (34) on the magnetizer/demagnetizer unit (30). The result is the aligned agglutinated particles held together by agglutination (36). In both of these embodiments, the positive binding reaction is detected not by the presence of agglutination, but by stabilization of the associated or “agglutinated” particles.

Generally, the agglutination reaction is permitted to proceed for as long as is necessary to stabilize the associations that result in binding of the particles to each other. Generally, the agglutination reaction is permitted to proceed for at least 15 minutes, preferably about 30 minutes, and more preferably about one hour. Stability can be detected by attempting to disrupt the agglutinated particles, e.g., by vibration, stirring, washing, or other disruptions of the solution in which the reaction has occurred.

There are a number of ways to detect particle agglutination, ranging from simple visual examination through sophisticated particle counting instrumentation (see PCT Publication No. WO 92/04469; Collet-Cassart et al., Clin. Chem. 1981, 27:64). The method of detection will depend on the sensitivity required.

In a specific embodiment, electrical or magnetic means can be used to detect agglutination. Packed particles, made of an electric insulating material, can form a block with high resistance to electric current flow. Such a block would not be possible with spherical particles as they would permit the free-flow of electrolytes between the open (uncontacted) surfaces. The degree of agglutination can be measured by measuring resistance or impedance. FIG. 11A illustrates a unit used to measure changes in impedance due to agglutination of electrically resistant geometrically regular particles (44). The unit consists of a meter to measure impedance (40), electrodes (41), wires to the electrodes from the meter to measure impedance (42), and electric field lines (43). Similarly, oriented magnetic particles can be easily distinguished from un-oriented particles by measuring the magnetic field they generate, or the interference they cause to an external magnetic field. In both cases, the degree of bonding orientation can be measured and quantitated.

Additionally, an optical means may be used to detect agglutination. FIG. 11B illustrates an optical transmitting and receiving unit(45) used to measure the occurrence of agglutination by measuring the intensity of light reflected from the fluorescent geometrically regularly shaped particles (48). The optical transmitting and receiving unit (45) consists of a transparent layer (46) and an opaque casing (47).

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all values are approximate, and are provided for description.

Patents, patent applications, publications, product descriptions, and protocols are cited throughout this application, the disclosures of which are incorporated herein by reference in their entireties for all purposes.

What is claimed:

1. A method for detecting low affinity binding of two molecules, which method comprises detecting agglutination of geometrically regular particles to which a molecule which is a member of a binding pair is covalently conjugated.

2. The method according to claim 1, wherein the geometrically regular particles have a shape selected from the group consisting of a triangular pyramid (tetrahedron), a cube, and a flat particle.

3. The method according to claim 1, wherein the particles have about the same volume.

4. The method according to claim 1, wherein the molecule conjugated to the particle is selected from the group consisting of an antibody, a ligand-binding site of a receptor, a substrate binding domain of an enzyme, and a carbohydrate binding domain of a lectin.

5. The method according to claim 1, wherein the particle is made from a material selected from the group consisting of plastic, glass, agarose, latex, starch or a starch derivative, silica, silicon, magnetic materials, and paramagnetic materials.

6. A composition comprising geometrically regular particles to which a molecule which is a member of a binding pair is covalently conjugated.

7. The composition of claim 6, wherein the geometrically regular particles have a shape selected from the group consisting of a triangular pyramid (tetrahedron), a cube, and a flat particle.

8. The composition of claim 6, wherein the particles have about the same volume.

9. The composition of claim 6, wherein the molecule conjugated to the particle is selected from the group consisting of an antibody, a ligand-binding site of a receptor, a substrate binding domain of an enzyme, and a carbohydrate binding domain of a lectin.

10. The composition of claim 6, wherein the particle is made from a material selected from the group consisting of plastic, glass, agarose, latex, starch or a starch derivative, silicon, silica, magnetic materials, and paramagnetic materials.

11. A method for preparing a composition comprising geometrically regular particles to which a molecule which is a member of a binding pair is covalently conjugated, which method comprises conjugating the molecule which is a member of a binding pair to particles of material extruded through a shaped die and cut at regular intervals so as to form geometrically regular particles of extruded material.

12. The method according to claim 11, wherein the shaped die is square.

13. The method according to claim 11, wherein the material is selected from the group consisting of plastic, glass, agarose, latex, starch or a starch derivative, silica, silicon, magnetic materials, and paramagnetic materials.

14. A method for preparing a composition comprising geometrically regular particles to which a molecule which is a member of a binding pair is covalently conjugated, which method comprises conjugating the molecule which is a member of a binding pair to particles of material molded so as to form geometrically regular molded particles.

15. The method according to claim 14, wherein the mold is cubic.

16. The method according to claim 14, wherein the material is selected from the group consisting of plastic, glass, agarose, latex, starch or a starch derivative, silica, silicon, magnetic materials, and paramagnetic materials.

17. A method for preparing a composition comprising geometrically regular particles to which a molecule which is a member of a binding pair is covalently conjugated, which method comprises conjugating the molecule which is a member of a binding pair to particles of material etched, micromachined, or laser cut from a block of the same material.

18. The method according to claim 17 wherein the particles are cubic.

19. The method according to claim 17 wherein the particles are flat.

20. The method according to claim 19 wherein the flat particles are joined.

21. The method according to claim 17, wherein the material is selected from the group consisting of plastic, glass, agarose, latex, starch or a starch derivative, silica, silicon, magnetic materials, and paramagnetic materials.
